(19) United States
(12) Patent Application Publication
(10) Pub. No.: US 2011/0301333 A1
(43) Pub. Date: Dec. 8, 2011

(54) REMOVAL OF PROTEIN AGGREGATES FROM BIOPHARMACEUTICAL PREPARATIONS USING CALCIUM PHOSPHATE SALTS

(75) Inventors: Ajish Potty, Woburn, MA (US); Alex Xenopolous, Billerica, MA (US)

(73) Assignee: Millipore Corporation, Billerica, MA (US)

(21) Appl. No.: 13/104,665

(22) Filed: May 10, 2011

Related U.S. Application Data
(60) Provisional application No. 61/397,229, filed on Jun. 8, 2010.

(51) Int. Cl. C07K 16/00; C07K 1/34; B01D 35/00; C07K 1/22

(52) U.S. Cl. 530/387.3; 530/413; 530/417; 530/388.1; 530/389.1; 530/387.1; 210/500.1

(57) ABSTRACT

The present invention provides novel and improved compositions containing calcium phosphate and methods of using the same for the removal of protein aggregates from biopharmaceutical compositions containing a product of interest, e.g., a therapeutic antibody or protein.

### CROSS-REFERENCE RELATED PATENT APPLICATIONS

This application claims the benefit of U.S. Provisional Patent Application No. 61/397,229, filed on Jun. 8, 2010 the entire contents of which are incorporated by reference herein.

### FIELD OF THE INVENTION

This invention relates to methods of removing protein aggregates from biopharmaceutical preparations containing a product of interest using a calcium phosphate salt.

### BACKGROUND OF THE INVENTION

Protein aggregates is one of the important impurities that needs to be removed from biopharmaceutical preparations containing a product of interest, e.g., a therapeutic protein or an antibody molecule. For example, protein aggregates and other contaminants must be removed from biopharmaceutical preparations containing a product of interest before the product can be used in diagnostic, therapeutic or other applications. Further, protein aggregates are also often found in antibody preparations harvested from hybridoma cell lines, and have to be removed prior to the use of the antibody preparation for its intended purpose. This is especially important in case of therapeutic applications and for obtaining Food and Drug Administration approval.

Removal of protein aggregates can be challenging as often there are similarities between the physical and chemical properties of protein aggregates and the product of interest in a biopharmaceutical preparation, which is often a monomeric molecule. There are many different methods in the art for the removal of protein aggregates from biopharmaceutical preparations including, for example, size exclusion chromatography, ion exchange chromatography and hydrophobic interaction chromatography.

Hydroxyapatite has been used in the chromatographic separation of proteins, nucleic acids, as well as antibodies. In hydroxyapatite chromatography, the column is normally equilibrated, and the sample applied, in a low concentration of phosphate buffer and the adsorbed proteins are then eluted in a concentration gradient of phosphate buffer (see, e.g., Giovannini, Biotechnology and Bioengineering 73:522-529 (2000)). However, in several instances, researchers have been unable to selectively elute antibodies from hydroxyapatite or found that hydroxyapatite chromatography did not result in a sufficiently pure product (see, e.g., Junbauer, J. Chromatography 476:257-268 (1989); Giovannini, Biotechnology and Bioengineering 73:522-529 (2000)).

Additionally, it has been observed and reported that packing most chromatography resins in a column is a laborious and an error-prone process. This is especially the case with ceramic hydroxyapatie (CHT) resin, a commercially available resin (available from BIORAD CORP), because of inherent fragility of the CHT resin particles. Further, while ceramic hydroxyapatite (CHT) has been used with some success for the removal of protein aggregates (see, e.g., U.S. patent publication no. WO/2005/044856), it is generally expensive and exhibits a low binding capacity for protein aggregates

### SUMMARY OF THE INVENTION

The present invention provides novel and improved compositions containing calcium phosphate salts (e.g., dicalcium phosphate anhydrous, dicalcium phosphate dehydrate, tricalcium phosphate and tetracalcium phosphate) and methods of using the same for the removal of protein aggregates from biopharmaceutical compositions containing a product of interest, e.g., a therapeutic antibody or protein.

The present invention provides, at least in part, for the use of a calcium phosphate salt (e.g., dicalcium phosphate anhydrous, dicalcium phosphate dehydrate, tricalcium phosphate and tetracalcium phosphate) for the removal of protein aggregates in a biopharmaceutical preparation containing a product of interest, where the salt is provided in a different format (e.g., a composite filter material) than previously known formats in the art (e.g., the CHT resin format sold by BIORAD CORP). The claimed compositions and methods result in a higher binding capacity for the protein aggregates than the prior art calcium phosphate based compositions and methods and are relatively inexpensive compared to the prior art compositions (e.g., a CHT resin format sold by BIORAD CORP), especially for use in chromatography methods. Further, the compositions of the present invention can be used as a disposable in a flow-through chromatography mode with broader operating pH ranges than the CHT resin format. A disposable format obviates the need to pack the compositions into a column, thereby simplifying the chromatography steps in a purification process and reducing the time it takes for the process.

In some embodiments, compositions and methods according to the present invention result in a purified sample containing less than 10%, or less than 5%, or less than 1% of protein aggregates. In some embodiments, a purified sample contains greater than 80%, or greater than 85%, or greater than 90%, or greater than 95% of the product of interest. In some embodiments, the improved compositions and methods of the claimed invention result in purified samples containing less than 1% of protein aggregates and/or greater than 95% of the product of interest.

In one aspect of the present invention, a method of separating a product of interest from protein aggregates in a biopharmaceutical preparation is provided, where the method comprises contacting the biopharmaceutical preparation with a composite filter material comprising a calcium phosphate salt, thereby to separate the product of interest from the protein aggregates.

In some embodiments, the composite filter material comprises a calcium phosphate salt selected from dicalcium phosphate anhydrous, dicalcium phosphate dehydrate, tricalcium phosphate and tetracalcium phosphate. In another embodiment, the calcium phosphate salt is hydroxyapatite.

In another aspect according to the present invention, a method of separating a product of interest from protein aggregates in a biopharmaceutical preparation is provided, where the method comprises the steps of: (a) providing a composite filter material comprising a calcium phosphate salt; (b) contacting the biopharmaceutical preparation with the composite filter material; and (c) eluting the product of interest from the composite filter material using a buffer comprising from 0 to 40 mM sodium phosphate and up to 1.5 M sodium chloride, thereby to separate the product of interest from protein aggregates. Further, non-phosphate buffers can also be used as shown with hydroxyapatite and other apatite materials (see, e.g., U.S. patent publication no. WO/2009/092010).

In yet another aspect according to the present invention, a method of separating a product of interest from protein aggregates in a biopharmaceutical preparation is provided, where the method comprises the steps of: (a) subjecting the biopharmaceutical preparation to a buffer exchange step using a suitable buffer; and (b) contacting the buffer exchanged preparation with a composite filter material comprising a calcium phosphate salt, where the protein aggregates bind to the composite filter material, thereby separating the protein aggregates from the product of interest.

In some embodiments of the various aspects of the present invention, the calcium phosphate salt is dicalcium phosphate anhydrous, dicalcium phosphate dehydrate, tricalcium phosphate or tetracalcium phosphate. In a particular embodiment, the calcium phosphate salt is hydroxyapatite.

In some embodiments of the aspects of the present invention, the product of interest is a therapeutic protein. In other embodiments, the product of interest is a therapeutic antibody. The antibody could be a monoclonal antibody, a polyclonal antibody, a humanized antibody, a chimeric antibody or an antigen binding fragment thereof. Also, the antibody could be an IgG, an IgM, an IgA, an IgD or an IgE antibody.

In some embodiments of the aspects of the present invention, the method of separating a product of interest from protein aggregates comprises using the calcium phosphate salt in a flow-through chromatography mode. In other embodiments, the method of separating a product of interest from protein aggregates comprises using the calcium phosphate salt in a bind and elute chromatography mode.

In case of the flow-through chromatography mode, the biopharmaceutical preparation containing the product of interest is treated to a buffer exchange step prior to contacting the preparation with a composite filter material containing a calcium phosphate salt, e.g., dicalcium phosphate anhydrous, dicalcium phosphate dehydrate, tricalcium phosphate and tetracalcium phosphate. In one embodiment, the calcium phosphate salt is hydroxyapatite.

In case of the bind and elute chromatography mode, the product of interest (e.g., a therapeutic protein or antibody) is eluted from the composite filter material containing a calcium phosphate salt using a buffer comprising from 0 to 40 mM sodium phosphate and up to 1.5M sodium chloride.

In some embodiments, a product of interest is eluted from the composite filter material using a buffer comprising from 1 to 20 mM sodium phosphate and from 0.2 to 2.5 M NaCl, and where the buffer has a pH from 6.4 to 7.6.

In various aspects of the present invention, a composite filter material according to the claimed invention comprises a calcium phosphate salt selected from the group consisting of dicalcium phosphate anhydrous, dicalcium phosphate dehydrate, tricalcium phosphate and tetracalcium phosphate. In a particular embodiment, the calcium phosphate salt is hydroxyapatite. In some embodiments, the calcium phosphate salt in a composite filter material, e.g., a hydroxyapatite composite filter material, binds protein aggregates with a higher capacity than a calcium phosphate salt in a resin format

In some embodiments according to the various aspects of the present invention, the product of interest subsequent to the separation methods described herein, contains less than 5%, or less than 4%, or less than 3%, or less than 2%, or less than 1% protein aggregates.

In some embodiments, the protein aggregates comprise high molecular weight aggregates.

In some embodiments according to the various aspects of the present invention, the methods and/or compositions of the present invention may be used in combination with one or more of Protein A chromatography, affinity chromatography, hydrophobic interaction chromatography, immobilized metal affinity chromatography, size exclusion chromatography, diafiltration, ultrafiltration, viral removal filtration, anion exchange chromatography, and/or cation exchange chromatography.

### BRIEF DESCRIPTION OF THE DRAWINGS

FIG. 1 depicts a representative chromatogram depicting the binding of a monoclonal antibody (mAb) to tri-calcium phosphate (TCP) in 5 mM sodium phosphate (NaPO₄), pH 6.5. The x-axis represents the volume passed through the column, the y-axis represents the absorbance at 280 nm and the secondary y-axis represents the conductivity in mS/cm. TCP was packed into a chromatography column with dimensions 3.8 cm×0.66 cm. Monomers eluted from a 20 column volume gradient of 5 mM sodium phosphate, pH 6.5 to 5 mM sodium phosphate+1 M NaCl, pH 6.5 and aggregates eluted from a step gradient in sodium phosphate concentration from 5 mM sodium phosphate+1 M NaCl, pH 6.5 to 400 mM sodium phosphate, pH 6.5. Solid lines represent absorbance readings and dotted lines represent conductivity readings.

FIG. 2 depicts a representative chromatogram depicting the binding of a monoclonal antibody (mAb) to dicalcium phosphate dihydrate (DCPD) in 5 mM sodium phosphate (NaPO₄), pH 6.5. The x-axis represents the volume passed through the column, the y-axis represents absorbance at 280 nm and the secondary y-axis is the conductivity in mS/cm. DCPD was packed into a chromatography column with dimensions 3.0 cm×0.66 cm. Monomers eluted from a 20 column volume gradient of 5 mM sodium phosphate, pH 6.5 to 5 mM sodium phosphate+1 M NaCl, pH 6.5 and aggregates eluted from a step gradient in sodium phosphate concentration from 5 mM sodium phosphate+1M NaCl, pH 6.5 to 200 mM sodium phosphate+1M NaCl, pH 6.5. Solid lines represent absorbance readings and dotted lines represent conductivity readings.

FIG. 3 depicts a representative chromatogram depicting the binding of a monoclonal antibody (mAb) to dicalcium phosphate anhydrous (DCPA) in 5 mM sodium phosphate (NaPO₄), pH 6.5. The x-axis represents the volume passed through the column, the y-axis represents absorbance at 280 nm and the secondary y-axis represents the conductivity in mS/cm. DCPA was packed into a chromatography column with dimensions 3.0 cm×0.66 cm. Monomers eluted from a 20 column volume gradient of 5 mM sodium phosphate, pH 6.5 to 5 mM sodium phosphate+1 M NaCl, pH 6.5 and aggregates eluted from a step gradient in sodium phosphate concentration from 5 mM sodium phosphate+1 M NaCl, pH 6.5 to 200 mM sodium phosphate+1 M NaCl, pH 6.5. Solid lines represent absorbance readings; dotted lines are conductivity readings.

FIG. 4 depicts a representative break through profile representing the operation of tri-calcium phosphate (TCP) in flow through mode. The x-axis represents the amount of aggregates loaded on to the column in mg of aggregates per ml of TCP and the y-axis represents the percentage of aggregates in the breakthrough pool. A 17% aggregate containing mAb in 10 mM sodium phosphate (NaPO₄)+1 M NaCl, pH 6.8, was passed through a TCP chromatography column with dimensions 3.8 cm×0.66 cm. 1 mL fractions were collected and analyzed for aggregate content using analytical SEC.

FIG. 5 depicts a comparison between a TCP column versus a TCP pad. A 17% aggregate containing mAb in 10 mM sodium phosphate (NaPO₄)+1 M NaCl, pH 6.8, was passed either through a TCP chromatography column with dimensions 3.8 cm×0.66 cm or through a 0.7 ml TCP pad. The x-axis represents the amount of aggregates loaded on to the column in mg of aggregates per ml of TCP and the y-axis represents the percentage of aggregates in the breakthrough pool. 1 mL fractions were collected and analyzed for aggregate content using analytical SEC. The percentage of aggregates is plotted as a function of the amount of aggregates loaded on to the column or pad. The open square symbols represent the break through profile for the TCP column while the closed squares represent the TCP pad.

FIG. 6 depicts the antibody aggregate break though profiles for CHT column (closed square), HA Pad (closed triangle), and TCP pad (closed circle). A 21% aggregate containing mAb in 5 mM sodium phosphate (NaPO₄)+0.5 M NaCl, pH 6.8, was passed either through a CHT chromatography column with dimensions 3.8 cm×0.66 cm. A 12% aggregate containing mAb in 10 mM sodium phosphate (NaPO₄)+0.65 M NaCl, pH 6.8, was passed either through a 0.7 ml HA pad. A 17% aggregate containing mAb in 10 mM sodium phosphate (NaPO₄)+1 M NaCl, pH 6.8, was passed either through a 0.7 ml TCP pad. 1 mL fractions were collected and analyzed for aggregate content using analytical SEC. The x-axis represents the amount of aggregates loaded on to the column in mg of aggregates per ml of column or pad and the y-axis represents the percentage of aggregates in the breakthrough pool.

### DETAILED DESCRIPTION OF THE INVENTION

In order that the present invention may be more readily understood, certain terms are first defined. Additional definitions are set forth throughout the detailed description.

### I. Definitions

The term “chromatography,” as used herein, refers to any kind of technique which separates the product of interest (e.g., a therapeutic protein or antibody) from contaminants and/or protein aggregates in a biopharmaceutical preparation.

The terms “flow-through process,” “flow-through mode,” and “flow-through chromatography,” as used interchangeably herein, refer to a product separation technique in which at least one product of interest (e.g., a therapeutic protein or antibody) contained in a biopharmaceutical preparation along with protein aggregates is intended to flow through a material (e.g., a composite filter material comprising a calcium phosphate salt such as, dicalcium phosphate anhydrous, dicalcium phosphate dehydrate, tricalcium phosphate and tetracalcium phosphate), where at least the protein aggregates bind the material. In a particular embodiment, the calcium phosphate salt is hydroxyapatite. In case of the flow-through chromatography mode according to the claimed invention, a biopharmaceutical preparation containing the product of interest is treated to a buffer exchange step prior to contacting the preparation with a composite filter material containing a calcium phosphate salt, e.g., dicalcium phosphate anhydrous, dicalcium phosphate dehydrate, tricalcium phosphate and tetracalcium phosphate. In a particular embodiment, the filter material comprises hydroxyapatite.

The terms “bind and elute mode” and “bind and elute process,” as used interchangeably herein, refer to a product separation technique in which at least one product of interest contained in a biopharmaceutical preparation binds to a material containing a calcium phosphate salt (e.g., a composite filter material containing a calcium phosphate salt, e.g., dicalcium phosphate anhydrous, dicalcium phosphate dehydrate, tricalcium phosphate and tetracalcium phosphate hydroxyapatite) along with protein aggregates and the product of interest is subsequently eluted from the material using a suitable elution buffer. In a particular embodiment, the composite material comprises hydroxyapatite. In case of the bind and elute chromatography mode according to the claimed invention, the product of interest (e.g., a therapeutic protein or antibody) is eluted from the composite filter material containing a calcium phosphate salt using a buffer comprising from 0 to 40 mM sodium phosphate and up to 1.5M sodium chloride. In some embodiments, a product of interest is eluted from the composite filter material using a buffer comprising from 1 to 20 mM sodium phosphate and from 0.2 to 2.5 M NaCl, and where the buffer has a pH from 6.4 to 7.6.

The terms “contaminant,” “impurity,” and “debris,” as used interchangeably herein, refer to any foreign or objectionable molecule, including a biological macromolecule such as a DNA, an RNA, one or more host cell proteins, endotoxins, lipids and one or more additives which may be present in a sample containing the product of interest that is being separated from one or more of the foreign or objectionable molecules. Additionally, such a contaminant may include any reagent which is used in a step which may occur prior to the separation process.

The term “immunoglobulin,” “Ig” or “antibody” (used interchangeably herein) refers to a protein having a basic four-polypeptide chain structure consisting of two heavy and two light chains, said chains being stabilized, for example, by interchain disulfide bonds, which has the ability to specifically bind antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of antibody light chains are referred to interchangeably as “light chain constant regions”, “light chain constant domains”, “CL” regions or “CL” domains. The “constant” domains of antibody heavy chains are referred to interchangeably as “heavy chain constant regions”, “heavy chain constant domains”, “CH” regions or “CH” domains. The “variable” domains of antibody light chains are referred to interchangeably as “light chain variable regions”, “light chain variable domains”, “VL” regions or “VL” domains. The “variable” domains of antibody heavy chains are referred to interchangeably as “heavy chain variable regions”, “heavy chain variable domains”, “VH” regions or “VH” domains.

Immunoglobulins or antibodies may be monoclonal or polyclonal and may exist in monomeric or polymeric form, for example, IgM antibodies which exist in pentameric form and/or IgA antibodies which exist in monomeric, dimeric or multimeric form. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fc and/or Fv fragments.

The term “antigen-binding fragment” refers to a polypeptide portion of an immunoglobulin or antibody that binds an antigen or competes with intact antibody (i.e., with the intact antibody from which they were derived) for antigen binding (i.e., specific binding). Binding fragments can be produced by recombinant DNA techniques, or by enzymatic or chemical cleavage of intact immunoglobulins. Binding fragments include Fab, Fab′, F(ab′)₂, Fv, single chains, and single-chain antibodies.

The term “biopharmaceutical preparation,” as used herein, refers to any composition containing a product of interest (e.g., a therapeutic protein or an antibody, which is usually a monomer) and unwanted components, such as protein aggregates (e.g., high molecular weight aggregates of the product of interest).

The term “protein aggregate” or “protein aggregates,” as used interchangeably herein, refers to an association of at least two molecules of a product of interest, e.g., a therapeutic protein or antibody. The association of at least two molecules of a product of interest may arise by any means including, but not limited to, covalent, non-covalent, disulfide, or nonreducible crosslinking.

The term “composite filter material,” as used herein, refers to a combination of an adsorbent material and a water-insoluble thermoplastic binder, which forms a porous, fixed bed of adsorbent material with suitable mechanical properties such as, for example, permeability, tensile strength and bending strength. In some embodiments, the composite filter material of the present invention comprises refined cellulose fibers, a calcium phosphate salt and a water soluble thermoset binder. In some embodiments, the calcium phosphate salt is dicalcium phosphate anhydrous, dicalcium phosphate dehydrate, tricalcium phosphate and tetracalcium phosphate. In a particular embodiment, the calcium phosphate salt is hydroxyapatite.

The term “chromatographic resin particles” as used herein, refers to adsorptive particles that are porous and have been manufactured to a diameter, approximately 20 to 100 micrometers, that makes them suitable for packing into a chromatographic column so that buffer can be flowed through at reasonable pressure drops.

### II. Exemplary Calcium Phosphate Salts for Use in the Claimed Compositions and Methods

Many different types of calcium phosphate salts can be used in the methods of the present invention. An exemplary calcium phosphate salt is hydroxyapatite, which has a chemical formula of (Ca)₁₀(PO₄)₆(OH)₂. In general, various exemplary calcium phosphate salts encompassed by the present invention can be represented by the general chemical formula: (Ca)ₓ(PO₄)_{y}. Several of these chemical phosphate salts differ from hydroxyapatite in structure. It has been previously reported that hydroxyapatite in a resin format can be used to remove impurities such as antibody aggregates from partially purified biopharmaceuticals. However, the present invention is based on the surprising and unexpected discovery that various other calcium phosphate salts having the general chemical formula of (Ca)ₓ(PO₄)_{y} can also be used for the removal of impurities such as antibody aggregates. Further, these salts can be used in a disposable pad format. Exemplary calcium salts encompassed by the present invention and represented by the general formula (Ca)ₓ(PO₄)_{y} include, but are not limited to, tricalcium phosphate (TCP where x is 3 and y is 2), dicalcium phosphate anhydrous (DCPA where x is 2 and y is 2), dicalcium phosphate dihydrate (DCPD where x is 2 and y is 2) and tetracalcium phosphate (where x is 4 and y is 2). It is also contemplated that instead of calcium, other divalent cations such as, for example, Mg²⁺ and the like, or combination thereof. may be used in the salts encompassed by the present invention

### III. Methods of Making Composite Filter Materials

In various embodiments according to the present invention, a calcium phosphate salt, as described herein, is in the form of a composite filter material. In some embodiments, a composite filter material according to the present invention is in a pad format. A number of methods known in the art as well as those described herein may be used for making such composite filter materials. For example, in an exemplary method, a calcium phosphate salt in water is blended with cellulose fibers and the mixture is passed through a funnel with filter paper. Once most of the material is de-wetted, the cellulose-salt pad is placed in an oven at 120° C. to evaporate the remaining water, thereby to form a composite filter material which is in a pad format. Once cooled, the pad can be cut into a desired size for placement into a desirable device.

In another method, a polyethylene is blended with a calcium phosphate salt and the mixture is subsequently poured into a desired mold, heated to a temperature suitable to melt the polyethylene and subsequently compressed, thereby to form a composite filter material in a pad format. In various embodiments, a composite filter material in pad format is referred to as a composite filter pad.

### IV. Flow-Through Chromatography Mode Using a Calcium Phosphate Salt

The composite filter materials comprising a calcium phosphate salt described herein may be used in a flow through chromatography mode. In general, flow though chromatography is routinely employed in protein purification processes. Typically, in the case of a flow through chromatography mode, a mixture of a product of interest and one or more impurities is passed through a chromatography column. Generally, the product of interest does not bind to the column, whereas the one or more impurities bind. The solution conditions that govern flow-through chromatography mode of operation can be obtained by screening various buffer compositions in a 96-well format as can be performed with chromatography resins. Typical flow through conditions may include an operating pH of 5.0-7.5, sodium phosphate from 0-100 mM, and sodium chloride from 0-1.5 M.

### VI. Bind and Elute Chromatography Mode Using a Calcium Phosphate Salt

The composite filter materials comprising a calcium phosphate salt, as described herein, may also be used in a bind and elute chromatography mode. In such a mode of operation, a mixture of product of interest and one or more impurities is passed through a chromatography column. However, the product of interest usually binds to the column, whereas most of the impurities do not bind. The solution conditions are subsequently altered to elute the product of interest. In instances where one or more impurities may also bind the column in addition to the product of interest, the product of interest is selectively eluted from the column while the one or more impurities remain bound to the column. The column may be later regenerated by cleaning with a buffer that elutes all of the impurities. The solution conditions for bind and elute mode of chromatography for calcium phosphate salts can be determined using high throughput screening of solution conditions in 96-well formats. The typical bind and elute operating conditions may include an operating pH of 5.0-7.5, sodium phosphate from 0-100 mM, and sodium chloride from 0-1.5 M.

In another exemplary embodiment, the composite filter material may be used in displacement chromatography mode, simulated moving bed, or other methods known to one skilled in the art.

### V. Methods for Detection of Aggregates

A number of methods are available to detect aggregates. The most commonly used method is analytical size exclusion chromatography. In this work, a dynamic light scattering instrument (Wyatt Technology) was used to detect aggregates for the 96-well plate high throughput screening experiments. Analytical SEC was used to analyze the fractions collected from column and composite filter pad experiments. Other dye-based methods for aggregate detection can also be used.

This invention is further illustrated by the following examples which should not be construed as limiting. The contents of all references, patents and published patent applications cited throughout this application, as well as the Figures, are incorporated herein by reference.

### EXAMPLES

### Example 1

### Removal of Monoclonal IgG Aggregates Using a β-Tricalcium Phosphate (TCP) Powder in Bind/Elute Mode

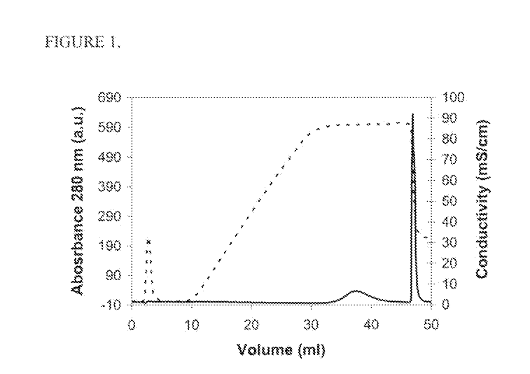

A partially purified monoclonal antibody (having a pI of ˜8.3) containing 50% aggregates was further purified using a column (3.8 cm×0.66 cm) packed with β-tricalcium phosphate (FLUIDINOVA, Moreira da Maia, Portugal). The column was equilibrated with 4 column volumes (CVs) of buffer 1 (0.4 M sodium phosphate, pH 6.5) at a flow rate of 350 cm/hr followed by 6 column volumes of buffer 2 (5 mM sodium phosphate, pH 6.5) at a flow rate of 350 cm/hr. About 500 μl of the partially purified mAb (at a concentration of 4 mg/ml) in 50 mM 2-(N-morpholino)ethanesulfonic acid (MES) buffer, 150 mM NaCl, pH 6.8, was loaded on the column at a flow rate of 175 cm/hr. The column was then washed with 4 column volumes of buffer 2. The mAb was subsequently eluted using a 20 column volume gradient of elution buffer (5 mM sodium phosphate, 1 M NaCl, pH 6.5). The column was regenerated with 5 column volumes of buffer 1. The UV absorbance (at 280 nm) and conductivity of the chromatograms were monitored through the entire purification run, as depicted in FIG. 1.

The elution and regeneration peaks were collected and analyzed for aggregate content using size exclusion chromatography (SEC) on a Zorbax GF450 column (AGILENT TECHNOLOGIES, Marlborough, Mass., USA) in 0.2 M sodium phosphate, pH 7.0 at 1 ml/min. The SEC data is summarized in Table 1. The monomer yield from this step was about 94%.

TABLE 1
Aggregate removal using TCP

| Sample | % Aggregates | % Monomer |
|---|---|---|
| Feed | 49.6 | 50.4 |
| Fraction 1 (elution peak) | 1.5 | 98.5 |
| Fraction 2 (regeneration peak) | 97.4 | 2.6 |

### Example 2

### Removal of Monoclonal Antibody Aggregates Using a Dicalcium Phosphate Dihydrate (DCPD) Powder in Bind/Elute Mode

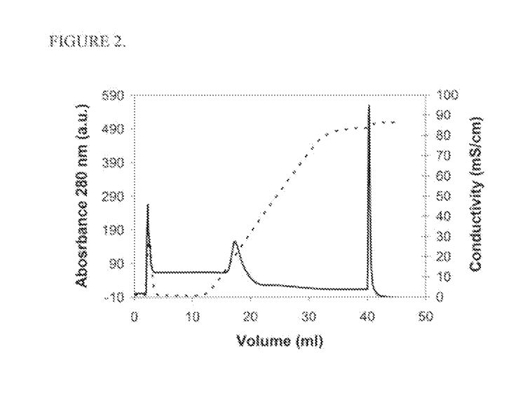

A partially purified monoclonal antibody (mAb) (pI of ˜8.3) containing 50% aggregates was further purified using a column (3.0 cm×0.66 cm) packed with dicalcium phosphate dihydrate (SPECTRUM CHEMICALS, Gardena, Calif., USA). The column was equilibrated with 6 column volumes of buffer 1 (0.4 M sodium phosphate, 1 M NaCl, pH 6.5) at a flow rate of 175 cm/hr followed by 6 column volumes of buffer 2 (10 mM sodium phosphate, pH 6.5) at a flow rate of 175 cm/hr. 500 μl of the partially purified mAb (at 4 mg/ml) in 50 mM MES, 150 mM NaCl, pH 6.8 was loaded on the column at a flow rate of 90 cm/hr. The column was then washed with 4 column volumes of buffer 2. The mAb was subsequently eluted using a 20 column volume gradient of elution buffer (10 mM sodium phosphate, 1 M NaCl, pH 6.5). The column was regenerated with 5 column volumes of buffer 1. The UV absorbance (at 280 nm) and conductivity of the chromatograms were monitored through the entire purification run, as depicted in FIG. 2.

### Example 3

### Removal of mAb Aggregates Using a Dicalcium Phosphate Anhydrous

(DCPA) Powder in Bind/Elute Mode

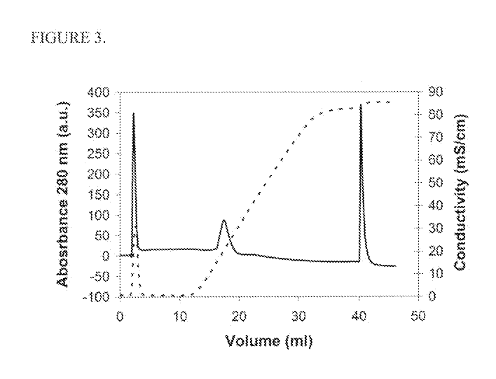

A partially purified monoclonal antibody (pI of ˜8.3) containing 54% aggregates was further purified using a column (3.0 cm×0.66 cm) packed with dicalcium phosphate anhydrous (SPECTRUM CHEMICALS, Gardena, Calif., USA). The column was equilibrated with 6 column volumes of buffer 1 (0.4 M sodium phosphate, 1 M NaCl, pH 6.5) at a flow rate of 175 cm/hr followed by 6 column volumes of buffer 2 (10 mM sodium phosphate, pH 6.5) at a flow rate of 175 cm/hr. About 500 μl of the partially purified mAb (at 4 mg/ml) in 50 mM MES, 150 mM NaCl, pH 6.8, was loaded on the column at a flow rate of 90 cm/hr. The column was then washed with 4 column volumes of buffer 2. The mAb was subsequently eluted using a 20 column volume gradient of elution buffer (10 mM sodium phosphate, 1M NaCl, pH 6.5). The column was then regenerated with 5 column volumes Of buffer 1. The UV absorbance (at 280 nm) and conductivity of the chromatograms were monitored through the entire purification run, as depicted in FIG. 3.

TABLE 2
Aggregate removal using DCPA in bind/elute mode

| Sample | % Aggregates | % Monomer |
|---|---|---|
| Feed | 54.1 | 45.9 |
| Fraction 1 (elution peak) | 17.5 | 82.5 |
| Fraction 2 (regeneration peak) | 95.2 | 4.8 |

### Example 4

### Removal of mAb Aggregates Using a β-Tricalcium Phosphate (TCP) Powder in Flow Through Mode

A partially purified monoclonal antibody (pI of ˜8.3) containing 12% aggregates was further purified using a column (2.0 cm×0.66 cm) packed with β-tricalcium phosphate (FLUIDINOVA, Moreira da Maia, Portugal). The column was equilibrated with 4 column volumes of buffer 1 (0.2 M sodium phosphate, pH 6.5) at a flow rate of 175 cm/hr followed by 6 column volumes of buffer 2 (10 mM sodium phosphate, pH 6.8) at a flow rate of 175 cm/hr. The solution conditions that resulted in optimal yield and aggregate removal were selected based on 96-well plate high throughput screening experiments.

About 11 mls of the partially purified mAb at 2 mg/ml in buffer 2 was loaded on the column at a flow rate of 58 cm/hr. The column was subsequently washed with 12 column volumes of buffer 2. The column was then regenerated with 5 column volumes of buffer 1.

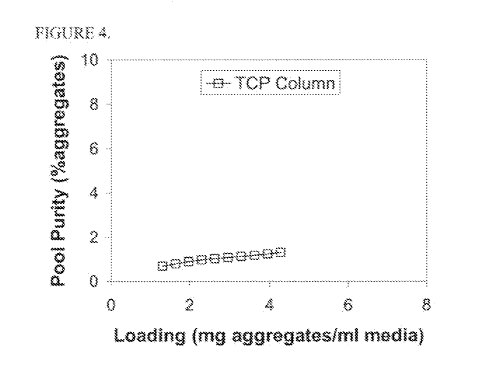

The UV absorbance (at 280 nm) and conductivity of the chromatograms were monitored throughout the purification run. The flow-through, wash, and regeneration fractions were collected to determine aggregate content and monomer yield. The yield was calculated to be about 98% based on the amount of antibody eluted from the regeneration cycle. FIG. 4 depicts the break through profile of aggregates for a TCP column in flow-through mode.

TABLE 3
Loading of aggregates on TCP column and purity of the flow-through pool

| | Loading (mg | |
|---|---|---|
| | aggregate/ml | Pool purity (% |
| Sample | media) | Monomer) |
| Feed | — | 88.0 |
| 1 | 1.31 | 99.3 |
| 2 | 1.64 | 99.2 |
| 3 | 1.98 | 99.1 |
| 4 | 2.31 | 99.0 |
| 5 | 2.64 | 98.9 |
| 6 | 2.96 | 98.9 |
| 7 | 3.29 | 98.9 |
| 8 | 3.62 | 98.8 |
| 9 | 3.95 | 98.8 |
| 10 | 4.28 | 98.7 |
| Regeneration | — | 15 |

### Example 5

### Procedure for Making Calcium Phosphate Salt-Cellulose Composite Filter

An exemplary method of making the composite filters according to the present invention is described in this Example.

An electronic weighing balance was used to determine the different weight ratios of the cellulose and salt (% solids), as depicted in Table 4 below.

TABLE 4
Composition of salt and cellulose for making composite filters

| | | Calcium Phosphate | Cellulose | |
|---|---|---|---|---|
| % Solids | Ratio | Salt (g) | pulp CSF, (g) | DI water (mls) |
| 18.75 | 80/20 | 15.0 | 3.75 | 487.5 |
| 18.75 | 70/30 | 13.125 | 5.625 | 487.5 |
| 18.75 | 60/40 | 11.25 | 7.50 | 487.5 |
| 18.75 | 50/50 | 9.375 | 9.35 | 487.5 |

A 1 liter graduated cylinder was filled with the appropriate volume of Milli-Q deionized water (MILLIPORE COPORATION, Billerica, Mass.) and was subsequently added to a blender containing cellulose. The cellulose was blended for about 60 seconds at a “High” setting and the salt was subsequently added to the blender for about 30 seconds at a “High” setting. A Buchner funnel was placed on a clamp stand and a vacuum tubing was placed onto the bottom of the funnel. A filter paper was inserted into the funnel base and wetted thoroughly with water. Subsequently, the vacuum source was turned on and vacuum pressure was applied to about 5 inches of mercury. The cellulose/salt mixture from the blender was poured into the funnel and the vacuum pressure was increased to about 15 inches of mercury, until most of the liquid was removed from the cellulose/salt mixture to form a pad. The funnel containing the pad was inverted on top of an adsorbent sheet and was shaken to separate the pad from the funnel. Following the removal of the pad from the funnel, another filter paper was placed into the funnel and wetted with deionized water. The pad was placed again into the funnel, such that the pad surface which was not initially in contact with the filter paper was now in contact with the paper and the pad was wetted thoroughly with deionized water. The vacuum was turned on and the pressure was increased to about 15 inches of mercury. The pad was removed and placed in a pre-heated drying oven at about 120° C. for several hours until completely dry. Once cooled, the pad can be cut into the desired size for placement into a device, such as, for example, a chromatography column.

### Example 6

### Removal of mAb Aggregates Using β-Tricalcium Phosphate (TCP) Cellulose Composite Filter in Flow Through Mode

A partially purified monoclonal antibody (pI of ˜8.3) containing about 17% aggregates was further purified using β-tricalcium phosphate-cellulose composite filter of 0.7 ml. The composite filter was equilibrated with 4 column volumes of buffer 1 (0.2 M sodium phosphate, pH 6.5) at a flow rate of 40 cm/hr followed by 6 column volumes of buffer 2 (5 mM sodium phosphate, 1 M NaCl, pH 6.8) at a flow rate of 40 cm/hr. The solution conditions that resulted in optimal yield and aggregate removal were selected based on 96-well plate high throughput screening experiments. About 10 mls of the partially purified mAb at a concentration of 2 mg/ml in buffer 2 was loaded on the composite filter at a flow rate of 13 cm/hr. The composite filter was subsequently washed with 12 column volumes of buffer 2. Following the wash, the composite filter was regenerated with 5 column volumes of buffer 1.

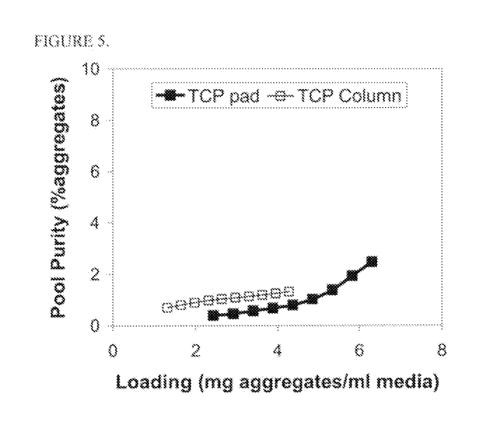

The UV absorbance (at 280 nm) and conductivity of the chromatograms were monitored throughout the entire purification run. The flow-through, wash, and regeneration fractions were collected to determine aggregate content and monomer yield. The yield was calculated to be about 90% based on the amount of antibody eluted from the regeneration cycle. FIG. 5 depicts a break through profile of aggregates for TCP column and TCP pad.

TABLE 5
Loading of IgG aggregates on TCP composite filter and purity of the
flow-through pool

| | Loading (mg | |
|---|---|---|
| | aggregate/ml | Pool purity (% |
| Sample | media) | Monomer) |
| Feed | — | 88.0 |
| 1 | 2.43 | 99.6 |
| 2 | 2.91 | 99.5 |
| 3 | 3.40 | 99.4 |
| 4 | 3.89 | 99.3 |
| 5 | 4.37 | 99.2 |
| 6 | 4.86 | 99.0 |
| 7 | 5.34 | 98.6 |
| 8 | 5.83 | 98.1 |
| 9 | 6.31 | 97.5 |
| Regeneration | — | 49.9 |

### Example 7

### Comparison of mAb Aggregates Removal Using Hydroxyapatite Chromatography Resin, Hydroxyapatite Cellulose Composite Filter, and β-Tricalcium Phosphate (TCP) Cellulose Composite Filter in Flow Through Mode

A partially purified monoclonal antibody (pI of ˜8.3) containing about 20% aggregates was further purified using a commercial hydroxyapatite chromatography resin (Ceramic hydroxyapatite) packed into a 1 ml column. The resin was equilibrated with 4 column volumes of buffer 1 (0.2 M sodium phosphate, pH 6.5) at a flow rate of 170 cm/hr followed by 6 column volumes of buffer 2 (5 mM sodium phosphate, 0.5 M NaCl, pH 6.8) at a flow rate of 90 cm/hr. About 10 mls of the partially purified mAb at a concentration of 2 mg/ml in buffer 2 was loaded on the composite filter at a flow rate of 90 cm/hr. The resin was subsequently washed with 12 column volumes of buffer 2. Following the wash, the resin was regenerated with 5 column volumes of buffer 1. The solution conditions that resulted in optimal yield and aggregate removal were selected based on 96-well plate high throughput screening experiments.

A partially purified monoclonal antibody (pI of ˜8.3) containing about 12% aggregates was further purified using a hydroxyapatite composite filter of 0.7 ml The composite filter was equilibrated with 4 column volumes of buffer 1 (0.2 M sodium phosphate, pH 6.5) at a flow rate of 40 cm/hr followed by 6 column volumes of buffer 2 (5 mM sodium phosphate, 1 M NaCl, pH 6.8) at a flow rate of 40 cm/hr. About 10 mls of the partially purified mAb at a concentration of 2 mg/ml in buffer 2 was loaded on the composite filter at a flow rate of 13 cm/hr. The composite filter was subsequently washed with 12 column volumes of buffer 2. Following the wash, the composite filter was regenerated with 5 column volumes of buffer 1. The solution conditions that resulted in optimal yield and aggregate removal were selected based on 96-well plate high throughput screening experiments.

A partially purified monoclonal antibody (pI of ˜8.3) containing about 17% aggregates was further purified using a hydroxyapatite composite filter of 0.7 ml The composite filter was equilibrated with 4 column volumes of buffer 1 (0:2 M sodium phosphate, pH 6.5) at a flow rate of 40 cm/hr followed by 6 column volumes of buffer 2 (10 mM sodium phosphate, 1 M NaCl, pH 6.8) at a flow rate of 40 cm/hr. About 10 mls of the partially purified mAb at a concentration of 2 mg/ml in buffer 2 was loaded on the composite filter at a flow rate of 13 cm/hr. The composite filter was subsequently washed with 12 column volumes of buffer 2. Following the wash, the composite filter was regenerated with 5 column volumes of buffer 1. The solution conditions that resulted in optimal yield and aggregate removal were selected based on 96-well plate high throughput screening experiments.

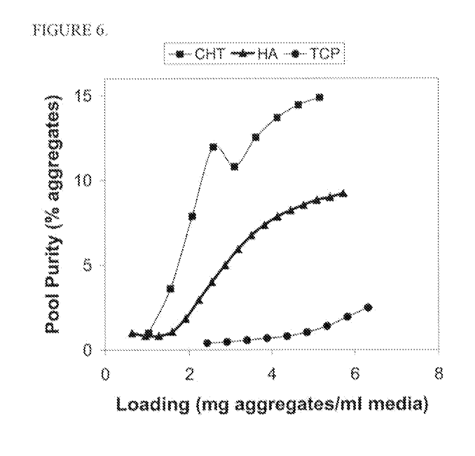

The UV absorbance (at 280 nm) and conductivity of the chromatograms were monitored throughout the entire purification run. The flow-through, wash, and regeneration fractions were collected to determine aggregate content and monomer yield. An overlay of the breakthrough profiles of the aggregates for the resin and the composite filters is depicted in FIG. 6. It was observed that the hydroxyapatite cellulose composite filter, and β-tricalcium phosphate-cellulose composite filter have 2- to 5-fold higher capacity and good selectivity (<2% aggregates in the pool) compared to commercial HA, as shown in FIG. 6.

The specification is most thoroughly understood in light of the teachings of the references cited within the specification which are hereby incorporated by reference. The embodiments within the specification provide an illustration of embodiments in this invention and should not be construed to limit its scope. The skilled artisan readily recognizes that many other embodiments are encompassed by this invention. All publications and inventions are incorporated by reference in their entirety. To the extent that the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

What is claimed is:

1. A method of separating a protein of interest from protein aggregates in a preparation, the method comprising contacting the preparation with a composite filter material comprising a salt comprising a divalent cation and a phosphate anion, thereby to separate the protein of interest from the protein aggregates.

2. The method of claim 1, wherein the salt is selected from the group consisting of dicalcium phosphate anhydrous, dicalcium phosphate dehydrate, tricalcium phosphate, tetracalcium phosphate, magnesium phosphate, and magnesium calcium phosphate,

3. The method of claim 1, wherein the protein of interest is separated using flow-through chromatography mode.

4. The method of claim 1, wherein the protein of interest is separated using bind and elute chromatography mode.

5. The method of claim 4, wherein the bind and elute mode is performed by eluting the protein of interest from the composite filter material using 0 to 40 mM sodium phosphate and up to 1.5M sodium chloride.

6. The method of claim 3, wherein the flow-through mode is performed by treating the preparation to a buffer exchange step prior to contacting the preparation with the composite filter material.

7. The method of claim 1, wherein the protein of interest is an antibody.

8. The method of claim 7, wherein the antibody is selected from the group consisting of a monoclonal antibody, a polyclonal antibody, a humanized antibody, a chimeric antibody and an antigen binding fragment thereof.

9. The method of claim 1, wherein the composite filter material is packed in a device.

10. The method of claim 7, wherein the antibody is an IgG, an IgM, an IgA, an IgD or an IgE antibody.

11. The method of claim 1, wherein the composite filter material is a disposable.

12. A method of separating a protein of interest from protein aggregates in a preparation, the method comprising the steps of:
(a) providing a composite filter material comprising a calcium phosphate salt;
(b) contacting the preparation with the composite filter material;
(c) eluting the protein of interest from the composite filter material using a buffer comprising 0 to 40 nM sodium phosphate and up to 1.5M sodium chloride,
thereby to separate the protein of interest from protein aggregates.

13. A method of separating a protein of interest from protein aggregates in a preparation, the method comprising the steps of:
(a) subjecting the preparation to a buffer exchange step using a suitable buffer; and
(b) contacting the buffer exchanged preparation with a composite filter material comprising a calcium phosphate salt,
wherein the protein aggregates bind to the composite filter material, thereby separating the protein aggregates.

14. The method of claim 12, wherein the calcium phosphate salt is selected from dicalcium phosphate anhydrous, dicalcium phosphate dehydrate, tricalcium phosphate and tetracalcium phosphate.

15. The method of claim 13, wherein the calcium phosphate salt is selected from dicalcium phosphate anhydrous, dicalcium phosphate dehydrate, tricalcium phosphate and tetracalcium phosphate.

16. A composite filter material comprising a calcium phosphate salt selected from dicalcium phosphate anhydrous, dicalcium phosphate dehydrate, tricalcium phosphate and tetracalcium phosphate, wherein the filter material binds protein aggregates with a higher capacity than a hydroxyapatite resin.
